(19) United States
(12) Patent Application Publication
(10) Pub. No.: US 2010/0063460 A1
(43) Pub. Date: Mar. 11, 2010

(54) DUAL-LUMEN NEEDLE

(76) Inventors: Jay Reed, Elk Grove Village, IL (US); Kevin Helle, Bartlett, IL (US)

Correspondence Address: GE HEALTHCARE, INC., IP DEPARTMENT 101 CARNEGIE CENTER, PRINCETON, NJ, 08540-6231, US

(21) Appl. No.: 12/515,553

(22) PCT Filed: Nov. 30, 2007
(86) PCT No.: PCT/US07/86022

§ 371 Date: May 20, 2009

Related U.S. Application Data
(60) Provisional application No. 60/867,919, filed on Nov. 30, 2006.

(51) Int. Cl. A61M 5/32

(52) U.S. Cl. 604/272

(57) ABSTRACT

A dual-lumen needle for aseptic filling and withdrawal of fluids from septum-plugged containers.

### FIELD OF THE INVENTION

The present invention is directed to the field of radiopharmaceutical handling equipment. More specifically, the present invention relates to a dual-lumen needle which allows the sterile transfer of pharmaceutical fluids.

### BACKGROUND OF THE INVENTION

A basic premise for the manufacture and handling of pharmaceutical fluids is the use of aseptic techniques. The pharmaceutical fluids are typically held within a container having a pierceable septum. The containers typically include a pharmaceutical type I glass bottle, a polymeric re-sealing septum, and an aluminum seal crimped onto the neck of the bottle and over the septum. These containers, depending on their function in the delivery chain of the pharmaceutical, are either meant to be filled with the pharmaceutical fluids or to supply the pharmaceutical fluid to another container or syringe or the like. Typically the components are cleaned and sterilized, then aseptically assembled, whereby the fluid is added to the bottle, the septa is inserted on the bottle, then the septa is capped and sealed with an aluminum seal. Alternatively, the components may be cleaned and sterilized, aseptically assembled with the drug product, and then the entire finished unit is terminally sterilized.

Whether the particular operation requires the pharmaceutical to be withdrawn from or added to the container, the art employs two needles for piercing the septum of the container. One needle is used for conducting the pharmaceutical into or out of the container. The other needle is used to either vent the container, ie, to allow the gas within the container to escape during filling, or to allow gas into the container to replace the void volumes created as the fluid is withdrawn. For example, sterile saline is often removed from one container to reconstitute freeze-dried pharmaceutical product in another container. Another example is the removal of Technetium-99m from one pharmaceutical container to reconstitute freeze-dried pharmaceutical product in another container. It is highly desirable that these pharmacy operations be performed aseptically.

Placement of two needles through the septum is difficult and fraught with risks to the pharmaceutical product and to the people handling it. One of the needles may be improperly positioned such that it hits the aluminum crimp, damaging both the crimp and/or the needle and causing a unit failure. If the open ends of the two needles are positioned in too close proximity to each other, liquid could transfer from one needle to the other and, especially when transferring a radiopharmaceutical fluid, cause a radioactive contamination event outside of the container and on or near personnel.

Another problem results from needles having a sharpened bevel causing tearing or coring of the septum, resulting in a unit failure. Additionally, should the septum core into the vent needle, thereby blocking it, can cause undetected overpressure in the filled container. The overpressure may cause the contents to spray out onto the pharmacist when drawing patient doses from the container. These problems also complicate the automation of such processes whereby additional handling equipment is required to precisely insert both needles through a septum.

The art has seen devices which could puncture a septum and provide both a fill path and a venting path, however, each of these devices cannot provide for aseptic fluid transfer whereby the septum integrity is retained after the fluid transfer operation.

For example, U.S. Pat. No. 7,091,494, which is commonly owned by the assignee of the instant invention, describes a dual-lumen spike for puncturing the septum of a container holding a radiopharmaceutical. The spike provides a first lumen for conducting fluid from the septum and a second lumen for venting the container. However, the large bore of a spike does not maintain the integrity of the septum after it has been withdrawn therefrom. The radiopharmaceutical remaining in the container would therefore be exposed to the outer environment.

Millipore Corporation manufactures and sells a dual needle concept under the tradename Sterisolutest. This product is used to remove finished pharmaceutical product from vials for sterility testing. Millipore has added a plastic fluid path for liquid transfer over the large bore needle, resulting in a very large bore requiring greater pressure and significant deformation of the septum in order to accomplish the fluid transfer. While it may be used in an aseptic operation, this device destroys the septum integrity such that the container would lose its hermetic seal at the conclusion of the process, thereby exposing the contents of the container to the outer environment.

Baxa manufactures a needle which provides venting by including plastic hub having axial grooves about the needle. Venting is accomplished by pushing the needle hub all the way into the septum such that the hub penetrates and open the septum, allowing gas to travel through the grooves in the plastic. This, however, is neither an aseptic operation, nor does it allow for verification of bacterial retention testing.

Wallace manufactures an oocyte recovery system using a dual lumen needle. One lumen is used to deliver a solution for internal lavage of the uterus. The other lumen is used to aspirate the solution in the recovery of oocytes. The needle is either 16 or 17 gauge and is 30-33 centimeters long. It includes 1 centimeter of echomarking at its free end. The length and construction of this needle preclude its use in aseptic filling operations, especially when conducting radiopharmaceuticals as there would be too much radioactive product retained within the needle itself.

The art would therefore benefit from a single needle which could both conduct fluid and vent the container in a manner that maintains the integrity of the septum of the container. The single needle would allow for aseptic filling and withdrawal of fluid into or out of the container. Such a needle would allow for the aseptic and terminal sterilization manufacture of pharmaceutical drug products into pre-sterilized, pre-sealed bottles. If purchased from a supplier, such a needle would eliminate the need for significant facility infrastructure, maintenance and monitoring, and validation of major systems in pharmaceutical plants, such as clean steam, water for injection, water for injection distribution systems, oil free compressed air, and facility clean-room HVAC.

### SUMMARY OF THE INVENTION

In view of the needs of the prior art, the present invention provides a dual-lumen needle which is capable of being inserted through the septum of a container plug to provide fluid transfer either into or out of the container while maintaining septum integrity after the fluid transfer operation and the needle is withdrawn from the septum.

### BRIEF DESCRIPTION OF THE DRAWINGS

FIG. 1 depicts a cross-sectional view of a dual-lumen needle of the present invention inserted into a container having an elastomeric septum.

FIG. 2 depicts an alternate embodiment of a dual-lumen needle of the present invention.

FIG. 3 depicts the area about the vent port of the dual-lumen needle of FIG. 2.

FIG. 4 depicts a cross-sectional view of the interior about the hub of the needle of FIG. 2.

FIG. 5 depicts a cross-section view of the interior of the hub of a dual-lumen needle where the gas-transferring lumen opens directly into the fluid-transferring needle.

FIG. 6 depicts a cross-sectional view of yet another dual-lumen needle of the present invention having a detachable filter element.

FIG. 7 depicts a cross-sectional view of still another dual-lumen needle of the present invention having a filter element incorporated into the needle hub.

### DETAILED DESCRIPTION OF THE PREFERRED EMBODIMENT

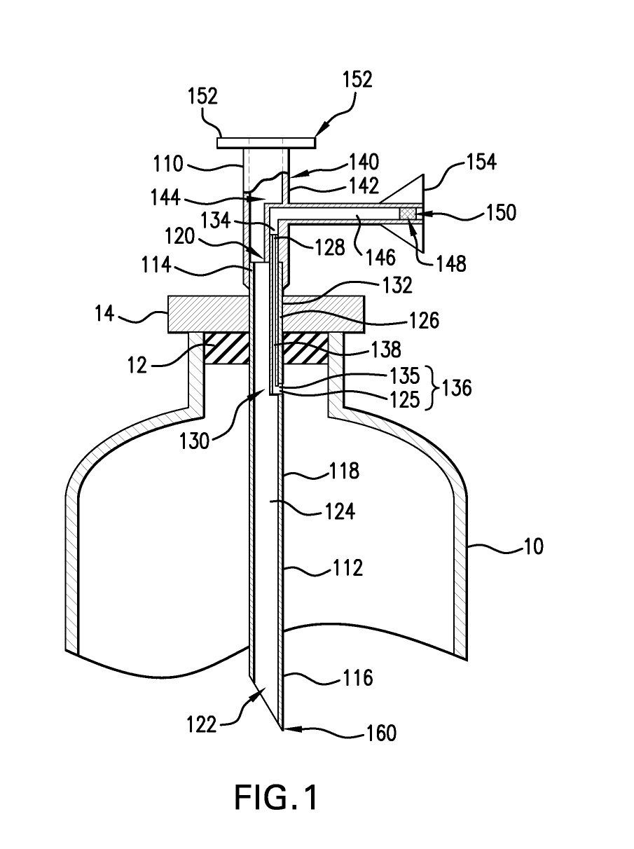

With reference to FIG. 1, the present invention provides a dual-lumen needle 110. Employing a tube-within-a-tube design, needle 110 allows the transfer of fluid into a container 10 having penetrable self-resealing septum 12 covered by a foil seal 14 using only a single needle penetration. Additionally, a small-bore, single penetration, will still leave a hermetically sealed container following the filling process. The present invention provides a single needle puncture during trans-septal fluid transfer. The needle is desirably fitted with a modified luer lock hub to allow for easy establishment and separation of the fluid and gas paths.

Referring still to FIG. 1, needle 110 includes a first elongate lumen 112 having opposed first and second open ends, 114 and 116, and an elongate first tubular body 118 extending therebetween. First open end 114 defines a hub port 120, second open end 116 defines a flow port 122, and tubular body 118 defines an elongate passageway 124 extending in fluid communication therebetween. Tubular body 118 further defines a vent opening 125 opening transversely to the longitudinal axis thereof. A second elongate lumen 126 includes opposed first and second ends 128 and 130 and an elongate second tubular body 132 extending therebetween. Open end 128 defines gas port 134, open end 130 defines vent aperture 135, and tubular body 132 defines an elongate passageway 138 extending therebetween. Vent aperture 135 and vent opening 125 of first lumen 112 are positioned in overlying registry so as to define a vent 136 in fluid communication with passageway 138 and the exterior of first lumen 112. The interface between vent opening 125 and vent aperture 135 is desirably sealed so as to prevent direct fluid communication between passageway 124 and passageway 138. The interface may be sealed by a biocompatible adhesive, solder, or any means known to those of skill in the art.

At least a portion of second lumen 122 is located within first passageway 124 of lumen 112. Needle 110 includes a hub 140 having a hub body 142 defining first and second open hub passageways 144 and 146 extending therethrough. First open hub passageway 144 extends in fluid communication with passageway 124 of first lumen 112. Second open hub passageway 146 extends in fluid communication with passageway 138 of second lumen 126. A filtration media 148 defining a gas passageway 150 therethrough is provided within second hub passageway 146. Gas passageway 150 of filtration media 148 is provided in fluid communication with passageway 138 of second lumen 126 so as to allow gas to flow therepast but not liquid. Hub 140 provides luer adaptor 152 for easy connection to and disconnection from other fluid conduits used in operation to provide a liquid flow through first lumen 112. Hub 140 optionally provides a second luer adaptor 154 so as to allow connection to conduit supporting filtration media providing finer particulate or bacterial separation.

Second end 116 of first lumen 112 includes a beveled tip 160 which further defines flow port 122. Tip 160 may be conventionally shaped to provide for ease of septum penetration in a manner that will allow the septum to sealingly engage tubular body 118 during fluid transfer and to then re-seal upon withdrawal of needle 110 and thereby maintain the sterility of the contents of the container which the septum seals. One penetration in a single manipulation maximizes the likelihood of success for aseptic transfers. Alternatively, the needle tip may be blunt-shaped or provide a transversely-opening flow port 122. The particular tip design may be selected so as to maximize the number of penetrations achievable by needle 110 prior to requiring replacement. Vent 136 is desirably provided to be longitudinally-spaced from flow port 122 so as to allow avoid the necessity of mixing between the gas flowing passageway 124 and fluid flowing through passageway 138.

Lumen 112 is desirably made from standard 316L stainless steel and may be nickel plated or plated with other metals based upon drug compatibility. Lumen 112 may be 16 gauge or smaller and desirably 17 gauge or smaller. Lumen 126 may be of identical materials as the primary lumen 112. Alternatively, the lumen 126 may be constructed of a biologically compatible plastic polymer. If lumen 126 is manufactured from a plastic polymer, it is possible to manufacture or mold the inner lumen and the hub concurrently. It is contemplated that the secondary tube has a cross-sectional area that is approximately 33% to 50% of the cross-sectional area of the primary lumen. Second lumen 126 is a smaller diameter tube than first lumen 112 and does not extend beyond the needle tip 160 and provides for gas transfer.

Needle hub 140 may be constructed of similar materials as currently utilized by needle manufacturers. The hub material should not leach into the pharmaceutical formulation or shed particulate. Making the retentive filter media 148 separately attachable to hub 140 allows for off-line integrity test verification of filter pore size prior to using with the needle of the present invention. Lumen 112 may be attached to the hub in a similar manner as needles are presently attached. It is desirable to increase the distance between the primary needle connection point and the luer fitting in order to accommodate the exit of the secondary lumen.

As it is possible for fluid to enter second lumen 126 during fluid transfer through first lumen 112, it is desirable to increase the diameter of gas flow path either at lumen 126, hub passageway 146, or at some section along its length prior to filtration media 148. Alternatively, the filter may be incorporated into the second end of the secondary lumen nearer the vent aperture so as to prevent liquid flow too far into passageway 138.

A hydrophobic filter may be utilized on the gas venting tube such that gasses can pass through, but are not wetted by aqueous solutions. The hydrophobic filter may be molded into the needle hub and act as a sterile gas vent. Alternatively, the hydrophobic filter may be attached to the terminal end of the gas vent tube. Alternatively still, the hydrophobic filter may be located some distance away affixed to flexible tubing attached to the needle hub. Non-aqueous solutions such as oils and alcohols may, conversely, utilize a similar design but with a hydrophilic filter as a vent. The filter desirably has a pore size that is bacterial retentive, such as 0.22 micron or 0.45 micron. Desirably, a common fitting such as a luer lock terminates the flexible tubing to allow for the attachment of a filter, either hydrophobic or hydrophilic as required.

During operation, the needles of the present invention typically provide a liquid through the first lumen as it extends further into a container than second lumen. It is desirable during either the delivery or removal of a liquid that there is no instance where the gas flow path is submerged in the liquid being delivered or removed. This assumes most operations will bring the needle in a downward direction into a container. However, in instances where the needle is directed in an upwards direction through a pierceable septum into a container, it may be desirable to deliver or remove fluid though the now-lower open end of the second lumen. It is contemplated that one or ordinary skill of the art will recognize that in such applications it may be desirable to provide a gas filtration media to filter fluid flow through the passageway of the appropriate needle.

Additionally, the needles of the present invention further allow an operator to pull a vacuum and back-fill a head space gas, such as N₂ without requiring a second puncture of the septum.

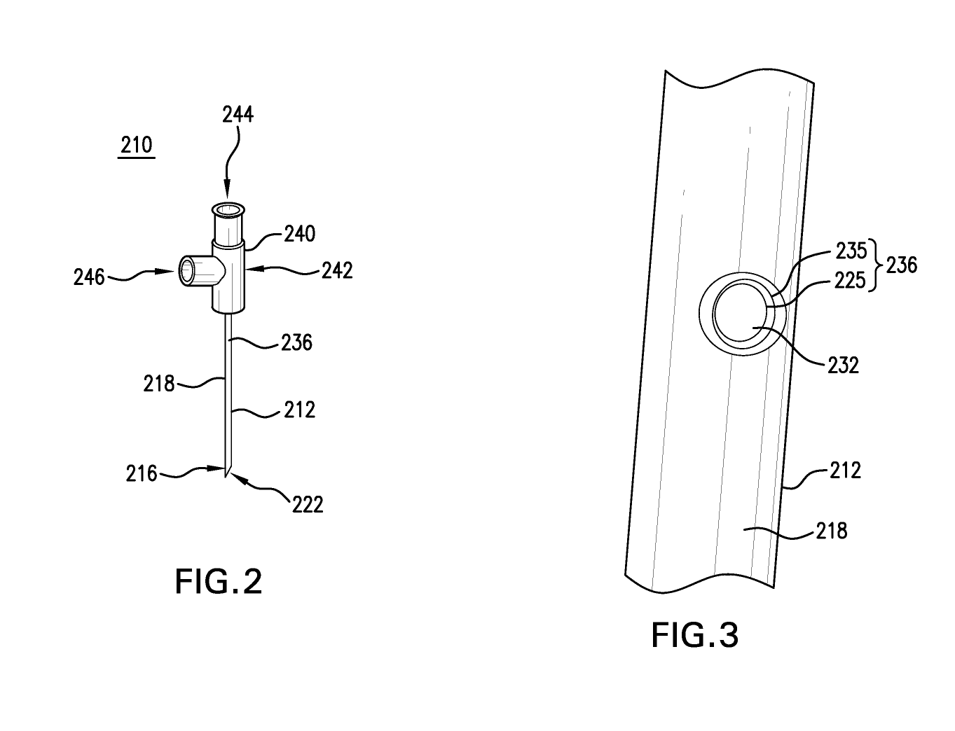

FIGS. 2-4 depicts an alternate dual-lumen needle 210 of the present invention. Needle 210 similarly includes a first lumen 212 having opposed first and second open ends, 214 and 216, and an elongate first tubular body 218 extending therebetween. First open end 214 defines a hub port 220, second open end 216 defines a flow port 222, and tubular body 218 defines an elongate passageway 224 extending in fluid communication therebetween. Tubular body 218 further defines a vent opening 225 opening transversely to the longitudinal axis thereof. A second elongate lumen 226 includes opposed first and second ends 228 and 230 and an elongate second tubular body 232 extending therebetween. Open end 228 defines gas port 234, open end 230 defines vent aperture 235, and tubular body 232 defines an elongate passageway 238 extending therebetween. Vent aperture 235 and vent opening 225 of first lumen 212 are positioned in overlying registry so as to define a vent 236 in fluid communication with passageway 238 and the exterior of first lumen 212. The interface, or seam, between lumens 212 and 226 about vent opening 225 and vent aperture 235 is desirably sealed so as to prevent direct fluid communication between passageway 224 and passageway 238. The interface may be sealed by a biocompatible adhesive, solder, or any means known to those of skill in the art.

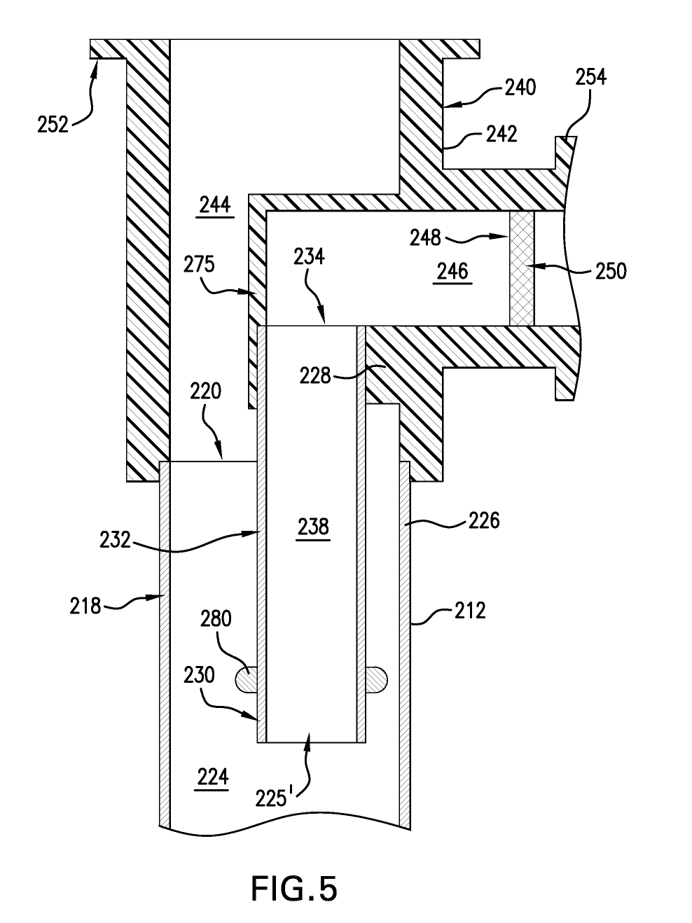

At least a portion of second lumen 222 is located within first passageway 224 of lumen 212. Needle 210 includes a hub 240 having a hub body 242 defining first and second open hub passageways 244 and 246 extending therethrough. First open hub passageway 244 extends in fluid communication with passageway 224 of first lumen 212. Second open hub passageway 246 extends in fluid communication with passageway 238 of second lumen 226. A filtration media 248 defining a gas passageway 250 therethrough is provided within second hub passageway 246. Gas passageway 250 of filtration media 248 is provided in fluid communication with passageway 238 of second lumen 226 so as to allow gas to flow therepast but not liquid. Hub 240 provides luer adaptor 252 for easy connection to and disconnection from other fluid conduits used in operation to provide a liquid flow through first lumen 212. As shown in FIG. 5, hub 240 optionally provides a second luer adaptor 254 so as to allow connection to conduit supporting filtration media providing finer particulate or bacterial separation.

FIG. 3 is a close-up view of vent 236 of needle 210. Tubular body 218 defines vent opening 235 which is in overlying registry with vent aperture 225 of second tubular body 232.

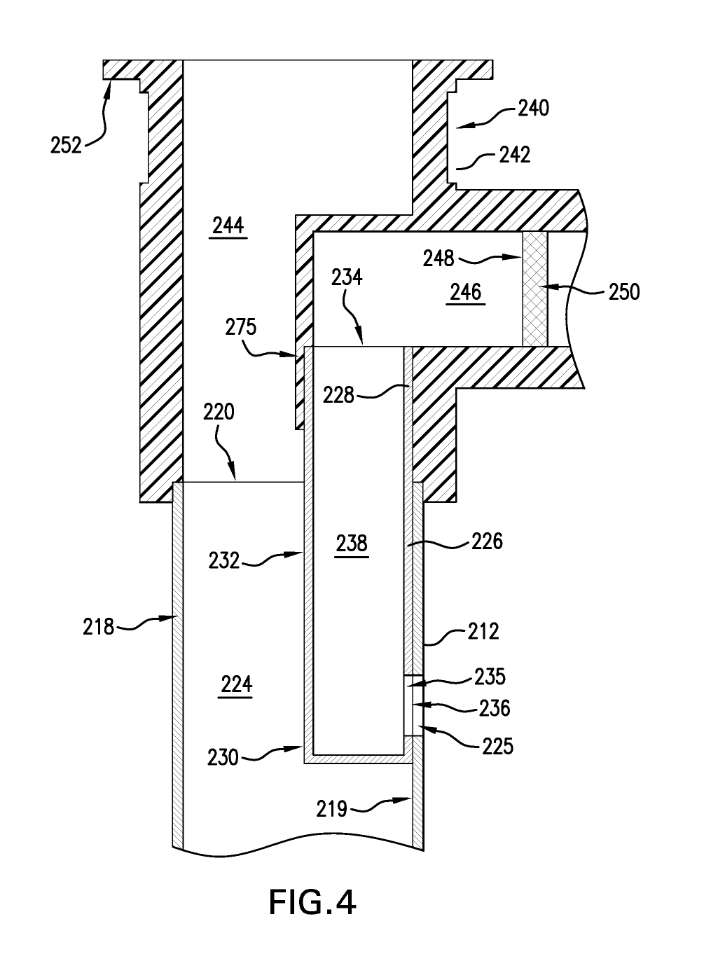

FIG. 4 shows more detail on how first and second lumens 212 and 226 are seated in hub body 242 so as to provide separate passageways for a fluid to be delivered through passageway 224 and a gas to be conducted through passageway 238. Hub body 242 includes a projecting wall 275 which annularly engages first end 228 of second lumen 226 so as to place gas port 234 in fluid communication with hub passageway 246.

Lumen 226 is shown in abutting engagement with the interior surface 219 of tubular body 218 of first lumen 212. It is contemplated by the present invention that when the lumens 212 and 226 are in such abutting engagement, that a single hole may be tapped through the lumens to form vent aperture 236 and vent opening

As shown in FIG. 5, it is further contemplated that second lumen 226 alternatively defines a vent aperture 225′ which opens in direct fluid communication with passageway 224 of first lumen 212. In this embodiment, first lumen 212 need not include a vent opening. Furthermore, second end 230 of second lumen 226 may be spaced from the interior surface of first lumen 212 so as to be freely supported within passageway 224. Second end 230 of second lumen 226 further supports an annular projection 280 extending into passageway 224 so as to impart turbulence to fluid flowing therepast towards flow port 222 of first lumen 212.

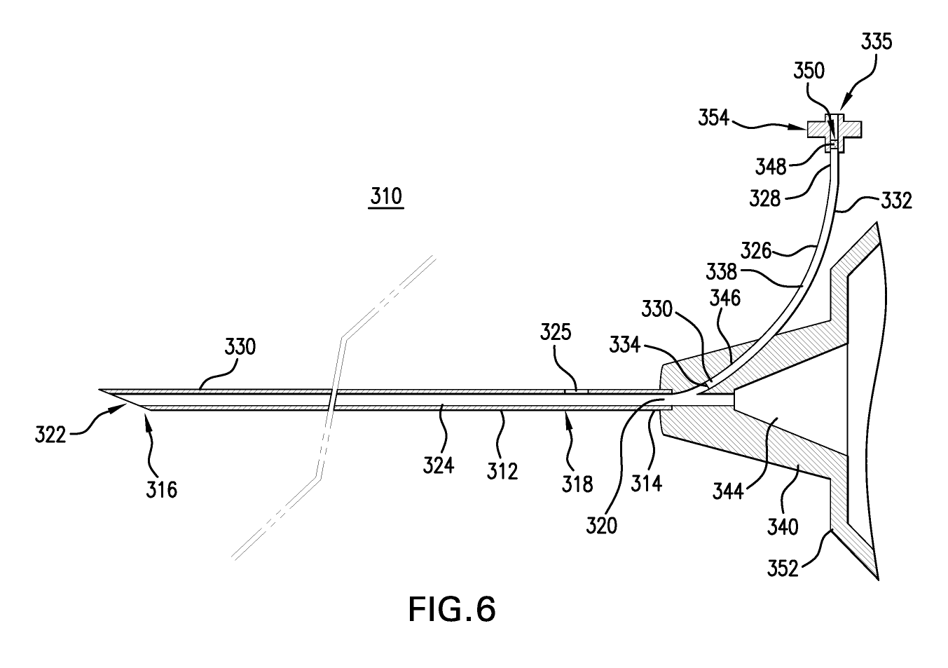

FIG. 6 depicts yet another dual-lumen needle 310 of the present invention having a filter element 348 incorporated into a flexible tube 326 inserted into the hub 340. Needle 310 includes a first lumen 312 having opposed first and second open ends, 314 and 316, and an elongate first tubular body 318 extending therebetween. First open end 314 defines a hub port 320, second open end 316 defines a flow port 322, and tubular body 318 defines an elongate passageway 324 extending in fluid communication therebetween. Tubular body 318 further defines a vent opening 325 opening transversely to the longitudinal axis thereof at a location adjacent to first open end 314.

A second elongate lumen 326 includes opposed first and second ends 328 and 330 and an elongate second tubular body 332 extending therebetween. Open end 330 defines gas port 334, open end 328 defines vent port 335, and tubular body 332 defines an elongate passageway 338 extending therebetween. At least a portion of second lumen 326 may be located within first passageway 324 of lumen 312. A filtration media 348 defining a gas passageway 350 therethrough spans across passageway 338 at second end 328 of lumen 326.

Needle 310 includes a hub 340 having a hub body 342 defining first and second open hub passageways 344 and 346 extending therethrough. First end 314 of lumen 312 is seated within hub 340 so that first open hub passageway 344 extends in fluid communication with passageway 324 of first lumen 312. Second lumen 326 extends through second open hub passageway 346 in fluid tight engagement with hub body 342 so that passageway 338 is in sealed fluid communication with passageway 324 of lumen 312. Whether permanently-affixed or removeably-affixed within second hub passageway 346, gas passageway 350 of filtration media 348 is thus provided in fluid communication with passageway 338 of second lumen 326 so as to allow gas to flow therepast but not liquid. Hub 340 provides luer adaptor 352 for easy connection to and disconnection from other fluid conduits used in operation to provide a liquid flow through first lumen 312. Second end 328 of lumen 326 supports a second luer adaptor 354 thereon so as to allow connection to other venting conduit if so desired. Providing filtration media within lumen 326 allows integrity and performance testing of filtration media 348 prior to mating lumen 326 to hub 340 as shown.

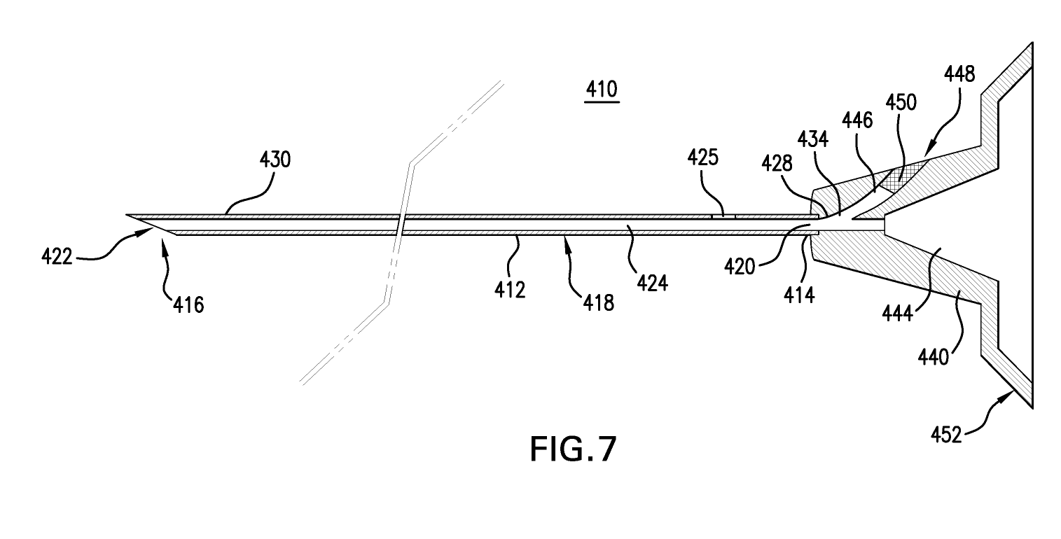

FIG. 7 depicts still another dual-lumen needle 410 of the present invention having a filter element 448 incorporated into the needle hub 440. Needle 410 similarly includes a first lumen 312 having opposed first and second open ends, 414 and 416, and an elongate first tubular body 418 extending therebetween. First open end 414 defines a hub port 420, second open end 416 defines a flow port 422, and tubular body 418 defines an elongate passageway 424 extending in fluid communication therebetween. Tubular body 418 further defines a vent opening 425 opening transversely to the longitudinal axis thereof at a location towards first end 414, but spaced therefrom sufficiently to ensure that vent opening 425 extends past the septum of the container into which needle 410 is inserted.

Needle 410 includes a hub 440 having a hub body 442 defining first and second open hub passageways 444 and 446 extending therethrough. First end 414 of lumen 412 is seated within hub 440 so that both first open hub passageway 444 and second open hub passageway 446 extend in fluid communication with passageway 424 of first lumen 412. A filtration media 448 defining a gas passageway 450 therethrough is provided within hub 440 spanning across second hub passageway 446.

While the particular embodiment of the present invention has been shown and described, it will be obvious to those skilled in the art that changes and modifications may be made without departing from the teachings of the invention. The matter set forth in the foregoing description and accompanying drawings is offered by way of illustration only and not as a limitation. The actual scope of the invention is intended to be defined in the following claims when viewed in their proper perspective based on the prior art.

What is claimed is:

1. A dual-lumen needle for delivering fluid comprising
a first elongate tubular body having opposed first and second open ends and defining an elongate first passageway extending therebetween;
a second elongate tubular body comprising opposed first and second open ends and defining a second elongate passageway extending therebetween, said first end of said second tubular body recessed from said first open end of said first tubular body;
a hub body supporting said first and second elongate tubular bodies, wherein said hub body defines a first hub passageway in direct fluid communication with said first passageway of said first tubular body and a second hub passageway in direct fluid communication with said second passageway of said second tubular body; and
filtration means defining a filtration passageway therethrough, said filtration passageway being in fluid communication with said second passageway of said second tubular body.

2. A dual-lumen needle of claim 1, wherein said second tubular body further comprises a first portion extending within said first passageway of said first tubular body.

3. A dual-lumen needle of claim 1, wherein said first open end of said second tubular body extends freely supported within first passageway.

4. A dual-lumen needle of claim 2, wherein said second tubular body extends freely within said first passageway.

5. The dual-lumen needle of claim 2, wherein said second hub passageway accommodates a portion of said second tubular body therein.

6. The dual-lumen needle of claim 1, wherein said second tubular body includes a protrusion on said elongate body for imparting turbulence to a fluid flowing therepast.

7. The dual-lumen needle of claim 6, wherein said protrusion is supported adjacent said first open end of said second tubular body.

8. The dual-lumen needle of claim 1, wherein said filtration media is supported within said second passageway.

9. The dual-lumen needle of claim 1, wherein said filtration media restricts fluid flow between said second passageway and a third fluid passageway located outside of said first tubular body.

10. The dual-lumen needle of claim 1, wherein said hub body further comprises luer-locking lugs projecting therefrom.

11. The dual-lumen needle of claim 1, wherein said second tubular body is flexible.

12. The dual-lumen needle of claim 6, wherein said at least one protrusion further comprises an annular collar extending about the second elongate tube.

13. A dual-lumen needle comprising:
a first elongate lumen comprising opposed first and second ends and an elongate first tubular body extending therebetween, said first tubular body defining an elongate passageway in fluid communication with said first and second open ends of said first lumen;
a second elongate lumen comprising opposed first and second ends and an elongate second tubular body extending therebetween, said second tubular body defining an elongate passageway in fluid communication with said first and second open ends of said second lumen, wherein at least a portion of said second lumen is located within said first passageway of said first lumen;
a hub comprising a hub body defining a first open hub passageway extending therethrough in fluid communication with said first passageway of said first lumen and a second open hub passageway extending therethrough in fluid communication with said second passageway of said second lumen; and
filtration media defining a gas passageway therethrough, said gas passageway in fluid communication with said second passageway of said second lumen.

14. A dual-lumen needle of claim 13, wherein said filtration media extends across said second passageway of said second lumen.

15. A dual-lumen needle of claim 13, wherein said filtration media extends across said second hub passageway.

16. A dual-lumen needle of claim 13, wherein said filtration media extends across said second passageway of said second lumen adjacent to one of said first and second ends of said second lumen.

17. A dual-lumen needle of claim 13, wherein said filtration media extends across a tubular passageway in fluid communication with said second hub passageway.

18. A dual-lumen needle of claim 17, wherein said tubular passageway is defined by a tube removably attached to said hub.

19. A dual-lumen needle of claim 13, wherein said second open end of said second lumen is in fluid communication with a vent port defined by said first tubular body of said first lumen.

20. A dual-lumen needle of claim 19, wherein said vent port opens transversely to the longitudinal axis of said first tubular body of said first lumen.

21. A dual-lumen needle of claim 19, wherein said vent port is located adjacent to said second open end of said first lumen.

22. A dual-lumen needle of claim 19, wherein said vent port is longitudinally-spaced from said second open end of said first lumen.

23. A dual-lumen needle of claim 19, wherein said first tubular body of said first lumen defines its corresponding second open end and said vent port to open transversely to the longitudinal axis of said first lumen.

24. A dual-lumen needle of claim 13, wherein said second end of said second lumen is positioned against said first tubular body.

25. A dual-lumen needle of claim 13, wherein said second end of said second lumen is freely supported within said first passageway of said first lumen.

26. A dual-lumen needle of claim 13, wherein said first tubular body of said first lumen has a cross-sectional dimension of a 16 gauge needle.

27. A dual-lumen needle of claim 13, wherein said first tubular body of said first lumen has a cross-sectional dimension less than a 16 gauge needle.

28. A dual-lumen needle of claim 13, wherein said second opening of said first tubular body opens in a direction substantially transverse to the longitudinal axis of said first tubular body.

29. A dual-lumen needle of claim 13, wherein said second opening of said first tubular body is axially-aligned with the longitudinal axis of said first tubular body.

30. A dual-lumen needle of claim 13, wherein said second tubular body supports a projection thereon extending into said first passageway of said first lumen.

31. A dual-lumen needle of claim 30, wherein said projection is located adjacent said second end of said second lumen.

32. A dual-lumen needle of claim 13, wherein at least one of said first end of said first lumen and said first end of said second lumen supports a luer fitting thereon.

33. A dual-lumen needle of claim 13, wherein said second lumen extends through said first open end of said first lumen.
